(19) United States
(12) Patent Application Publication
(10) Pub. No.: US 2006/0025484 A1
(43) Pub. Date: Feb. 2, 2006

(54) Use of 1-(2-chloroethyl)-1-nitroso-3-(2-hydroxyethyl)urea for treatment of pancreatic carcinomas, soft tissue sarcomas, testicular tumors, lymphomas, thymomas, wilms' tumors, renal carcinomas, melanomas, lung tumors, intracerebral metastases, tumors in the head and neck area and mammary carcinomas

(75) Inventor: Gerhard Eisenbrand, Heidelberg (DE)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: Faustus Forschungs CIE. Translational Cancer Research GmbH

(21) Appl. No.: 11/210,313

(22) Filed: Aug. 24, 2005

Related U.S. Application Data
(63) Continuation of application No. PCT/EP04/01875, filed on Feb. 25, 2004.

(30) Foreign Application Priority Data
Feb. 25, 2003 (DE) 103 07 928.9

(51) Int. Cl. A61K 31/175

(52) U.S. Cl. 514/589

(57) ABSTRACT

This invention relates to the use of 1-(2-chloroethyl)-1-nitroso-3-(2-hydroxyethyl)urea (hereinafter also referred to as HECNU) for treatment of pancreatic carcinomas, soft tissue sarcomas, testicular tumors, lymphomas, thymomas, Wilms' tumors, renal carcinomas, melanomas, lung tumors, intracerebral metastases, tumors in the head and neck area and mammary carcinomas.

### CROSS-REFERENCE TO RELATED APPLICATIONS

This application is a continuation of International Application No. PCT/EP2004/001875, filed Feb. 25, 2004, which was published in the German language on Sep. 10, 2004, under International Publication No. WO 2004/075887 A1 and the disclosure of which is incorporated herein by reference.

Use Of 1-(2-Chloroethyl)-1-Nitroso-3-(2-Hydroxyethyl)Urea For Treatment Of Pancreatic Carcinomas, Soft Tissue Sarcomas, Testicular Tumors, Lymphomas, Thymomas, Wilms' Tumors, Renal Carcinomas, Melanomas, Lung Tumors, Intracerebral Metastases, Tumors In The Head And Neck Area And Mammary Carcinomas

### BACKGROUND OF THE INVENTION

This invention relates to the use of 1-(2-chloroethyl)-1-nitroso-3-(2-hydroxyethyl)urea (also referred to below as HECNU) for treatment of pancreatic carcinomas, soft tissue sarcomas, testicular tumors, lymphomas, thymomas, Wilms' tumors, renal carcinomas, melanomas, lung tumors, intracerebral metastases, tumors in the head and neck area and mammary carcinomas.

It is known that bis-(2-chloroethyl)-1-nitrosourea (hereinafter also referred to as BCNU) can be used in the treatment of various types of cancer. BCNU is lipophilic but it has a low solubility in water, so that additional solvents such as ethanol must be used in application. Furthermore, it has been found that BCNU has a high toxicity for bone marrow and the lungs and can also cause pulmonary fibroses. There has therefore been a search for alternatives to BCNU.

Various asymmetrical 1,3-disubstituted nitrosoureas such as HECNU have been synthesized, as described, for example, in German Patent DE 26 23 420 and U.S. Pat. No. 4,150,146. The antineoplastic effect of these compounds has been tested in two animal models (rat leukemia L 5222 and s.c. implanted Walker carcinosarcoma).

In addition, clinical phase II studies with 1-(2-chloroethyl)-3-(2-hydroxyethyl)-1-nitrosourea (HECNU) have been conducted on patients with advanced cancer of the stomach or large intestine, as described by H. H. Fiebig et al. in Contrib. Oncol., vol. 37, pp. 157-162 (1989). In treatment of gastric carcinomas, a partial remission was observed in 31% of the patients treated, whereas only marginal activities and response rates have been demonstrated in colorectal carcinoma.

In addition, clinical phase II studies with HECNU for treatment of malignant brain tumor have also been conducted, as described by H. Henss et al. in Contrib. Oncol., vol. 37, pp. 163-167 (1989). A significant antineoplastic activity of HECNU in malignant brain tumors was found in that study.

Clinical phase II studies with HECNU for treatment of malignant supratentorial gliomas have also been conducted, as described by P. Georges et al. in J. Neuro-Oncology, 6, 211-219 (1988). Here again, HECNU demonstrated its activity. The more favorable toxicity profile in comparison with other nitrosoureas is emphasized in particular.

In addition, clinical phase II studies have also been conducted with HECNU for treatment of recurrent malignant gliomas by intra-arterial and infraophthalmic infusion, as described by M. Poisson et al. in J. Neuro-Oncology, 8, 255-262 (1990). Again, only the field of neuro-oncology is included in this publication.

It is thus known that HECNU (1-(2-chloroethyl)-1-nitroso-3-(2-hydroxyethylurea)) has a high activity for tumors of the CNS as well as tumors of the stomach as indications. Furthermore, the low efficacy in colon cancer has also been described.

In addition to the types of cancer mentioned above, however, there is also an increasing demand for successful treatment of other types of cancer, namely pancreatic carcinomas, soft tissue sarcomas, testicular tumors, lymphomas, thymomas, Wilms' tumors, renal carcinomas, melanomas, lung tumors, intracerebral metastases, tumors in the head and neck area and mammary carcinomas.

### BRIEF SUMMARY OF THE INVENTION

Therefore, the object of this invention is to treat pancreatic carcinomas, soft tissue sarcomas, testicular tumors, lymphomas, thymomas, Wilms' tumors, melanomas, lung tumors, intracerebral metastases, tumors in the head and neck area and mammary carcinomas.

It has surprisingly now been found that HECNU is suitable for use for treatment of pancreatic carcinomas, soft tissue sarcomas, testicular tumors, lymphomas, thymomas, Wilms' tumors, renal carcinomas, melanomas, lung tumors, intracerebral metastases, tumors in the head and neck area and mammary carcinomas.

Use of HECNU is preferred for treatment of pancreatic carcinomas, soft tissue sarcomas, lymphomas, thymomas, renal carcinomas, melanomas, lung tumors, intracerebral metastases and tumors in the head and neck area.

Use of HECNU is especially preferred for treatment of pancreatic carcinomas, soft tissue sarcomas, lymphomas, renal carcinomas, melanomas, intracerebral metastases and tumors in the head and neck area.

Use of HECNU is most especially preferred for treatment of pancreatic carcinomas, lymphomas, melanomas and tumors in the head and neck area.

In vitro experiments, animal experiments and clinical studies have shown that HECNU also has a high activity in treatment of these cancers.

HECNU has the following structure and can be synthesized by methods such as those disclosed in German Patent DE 26 23 420 and U.S. Pat. No. 4,150,146, as described above.

In comparison with BCNU, HECNU has a greatly reduced bone marrow toxicity, pulmonary toxicity and renal toxicity. It is assumed that this is attributable to the fact that when HECNU is used, there cannot be any carbamoyl reactions in the body because no carbamoylating metabolite is formed in its degradation in the body. Furthermore, HECNU does not inhibit glutathione reductase in the lungs. It is also a much weaker carcinogen than BCNU.

Because of the good water solubility of HECNU, this compound can also be administered by simple injection.

In addition, HECNU has only a low gastrointestinal toxicity and thus causes very little nausea and vomiting in patients and is also tolerated better.

This invention also relates to the use of HECNU for the preparation of a pharmaceutical drug for treatment of pancreatic carcinomas, soft tissue sarcomas, testicular tumors, lymphomas, thymomas, Wilms' tumors, renal carcinomas, melanomas, lung tumors, intracerebral metastases, tumors in the head and neck area and mammary carcinomas.

This invention also relates to a method for therapeutic and/or prophylactic treatment of a mammal requiring treatment by administration of HECNU for treatment of pancreatic carcinomas, soft tissue sarcomas, testicular tumors, lymphomas, thymomas, Wilms' tumors, renal carcinomas, melanomas, lung tumors, intracerebral metastases, tumors in the head and neck area and mammary carcinomas.

### DETAILED DESCRIPTION OF THE INVENTION

For treatment of the cancers listed above, HECNU is especially preferably administered intravenously but also intramuscularly, intraperitoneally, subcutaneously or orally. External application is also possible. It is preferably administered through intravenous injection or intravenous infusion.

HECNU may be used according to this invention in any suitable formulation under the prerequisite that the development and maintenance of adequate levels of the active ingredient are ensured. This may be accomplished, for example, by oral or parenteral administration in suitable doses. The pharmaceutical preparation of the active ingredient is advantageously in the form of unit doses which are tailored to the required administration. For example, a unit dose may be a tablet, a pill, a capsule, a suppository or a measured volume of a powder, granules, a solution, an emulsion or a suspension.

The term “unit dose” in the sense of the present invention is understood to refer to a physically defined unit which contains an individual amount of an active ingredient in combination with a pharmaceutical vehicle and whose active ingredient content corresponds to a fraction or a multiple of a single therapeutic dose. A single dose preferably contains the amount of active ingredient administered at one time, usually corresponding to an entire daily dose or one-half, one-third or one-fourth of a daily dose. If only a fraction, such as one-half or one-third of the unit dose, is needed for a single therapeutic administration, then the unit dose is advantageously suitable for division, e.g., in the form of a scored tablet.

The inventive use of HECNU in a suitable pharmaceutical drug may be performed with approximately 0.1 to 500 mg, preferably 10 to 200 mg and especially 50 to 150 mg active ingredient if administered in unit doses intended for administration to humans, for example.

In general, the active ingredient(s) is/are used in daily dose of 0.1 to 5 mg/kg body weight, preferably 1 to 3 mg/kg in human medicine, optionally in the form of multiple individual doses, preferably 1 to 3, to achieve the desired results. A single dose contains the active ingredient(s) in amounts of 0.1 to 5 mg/kg body weight, preferably 1 to 3 mg/kg. Similar doses may be used in oral treatment.

The inventive therapeutic use of HECNU in a pharmaceutical drug may be performed one to four times a day at fixed times or at variable times, e.g., before meals and/or in the evening. However, it may be necessary to depart from the aforementioned dosages, namely as a function of the type, weight and age of the individual to be treated, the type and severity of the disease, the type of preparation and the form of administration of the pharmaceutical drug as well as the period of time, i.e., interval within which it is administered. Thus in some cases it may be sufficient to use less than above-mentioned amount of active ingredient, whereas in other cases the amount of active ingredient indicated above must be exceeded. It may also prove expedient to administer the pharmaceutical drug only once a day or at intervals of several days.

Anyone skilled in the art can determine on the basis of his or her technical expertise the required optimum dosage and form of administration of HECNU.

The dose of HECNU administered to the patient especially preferably amounts to 60 to 200 mg/m² (based on the body surface area of the patient, preferably approximately 80 to approximately 150 mg/m², especially preferably approximately 100 to approximately 120 mg/m².

The inventive use of HECNU may be in the form of pharmaceutical drugs which usually include HECNU and nontoxic pharmaceutically acceptable vehicles which are used as a mixture or diluent, e.g., in solid, semisolid or liquid form or as a containment, e.g., in the form of a capsule, a tablet coating, a bag or some other container for the therapeutically active component. A vehicle may be used for example as a mediator for uptake of the pharmaceutical drug by the body, as a formulation aid, as a sweetener, as a taste corrector, as a coloring agent or as a preservative.

For oral administration, for example, tablets, coated pills, hard and soft capsules, e.g., of gelatin, dispersible powders, granules, aqueous and oil-based suspensions, emulsions, solutions or syrups may be used.

Tablets may contain inert diluents, e.g., calcium carbonate, calcium phosphate, sodium phosphate or lactose; granulation aids and distribution aids, e.g., cornstarch or alginates; binders, e.g., starch, gelatin or acacia gum; and lubricants, e.g., aluminum stearate or magnesium stearate, talc or silicone oil. They may also be provided with a coating, which may be such that it delays the release and absorption of the pharmaceutical preparation in the gastrointestinal tract so that better tolerability and protracted or delayed release are achieved. Gelatin capsules may contain the drug in mixture with a solid diluent, e.g., calcium carbonate or kaolin, or an oil-based diluent such as olive oil, peanut oil or paraffin oil.

Aqueous suspensions may contain suspension agents, e.g., sodium carboxymethyl cellulose, methylcellulose, hydroxypropyl cellulose, sodium alginate, polyvinylpyrrolidone, gum tragacanth or acacia gum; dispersants and wetting agents, e.g., polyoxyethylene stearate, eptadecaethyleneoxycatanol, polyoxyethylenesorbitol monooleate or lecithin; preservatives, e.g., methyl hydroxybenzoate or propyl hydroxybenzoate; taste correctors; sweeteners, e.g., sucrose, lactose, sodium cyclamate, dextrose, invert sugar syrup.

Oil-based suspensions may contain for example peanut oil, olive oil, sesame oil, coconut oil or paraffin oil and thickeners, e.g., beeswax, hard paraffin or cetyl alcohol as well as sweeteners, taste correctors and antioxidants.

Water-dispersible powders and granules may be included in the inventive use of HECNU in mixture with dispersants, wetting agents and suspension agents, e.g., those listed above, and with sweeteners, taste correctors and coloring agents.

Emulsions may contain olive oil, peanut oil or paraffin oil, for example, in addition to emulsifiers such as acacia gum, gum tragacanth, phosphatides, sorbitan monooleate, polyoxyethylene sorbitan monooleate and sweeteners and taste correctors.

Aqueous solutions may contain preservatives, e.g., methyl hydroxybenzoate or propyl hydroxybenzoate; thickeners; taste correctors; sweeteners, e.g., sucrose, lactose, sodium cyclamate, dextrose, invert sugar syrup and taste correctors and coloring agents.

For parenteral use of the pharmaceutical drugs, sterile injectable aqueous solutions, isotonic saline solutions or other solutions may be used.

This invention is explained in greater detail below on the basis of examples.

### EXAMPLES

Pancreatic carcinoma
Clinical study:
Phase II: ¼ partial remission (PR) (44 weeks)

+1 minor response (MR) (23 weeks)

| Tumor | Karnofsky | Previous | Tumor | | | Time to P |
|---|---|---|---|---|---|---|
| origin | index, % | chemotherapy | manifestation | Dose, mg/m² | Effect | (weeks) |
| Pancreas | 60 | 21 × FU | Liver, | 100 | PR | 44 |
| | | | primary TU | | | |
| Pancreas | 90 | 4 × ACO, | Liver | 100 | MR | 23 |
| | | 2 × FU | | | | |

In a clinical phase II study, a partial remission was observed in one of four treated patients with pancreatic cancer. A minor response was obtained in another patient.

Inhibiting effects in the low μg/mL range were also found in in-vitro experiments conducted with various pancreas lines, an inhibiting effect normally being achieved with an HECNU concentration of less than 10 μg/mL.

In in-vitro experiments, an inhibiting effect was also achieved on tumors of the head and neck.

Furthermore, the activity of HECNU has also been investigated in a number of subcutaneously implanted human tumor xenograft models in the naked mouse. In a representative spectrum of tumors, good to very good inhibiting effects on tumor growth were found on these human xenograft tumor models in the naked mouse. These included soft tissue sarcomas, testicular tumors, thymomas, melanomas, mammary carcinomas, tumors of the lung, Wilms' tumors and renal carcinomas.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A method for treating pancreatic carcinomas, soft tissue sarcomas, testicular tumors, lymphomas, thymomas, Wilms' tumors, renal carcinomas, melanomas, lung tumors, intracerebral metastases, tumors in the head and neck area and/or mammary carcinomas, the method comprising:
administering an effective amount of 1-(2-chloroethyl)-1-nitroso-3-(2-hydroxyethyl)urea (HECNU) to a patient in need of such treatment.

2. A method for producing a pharmaceutical drug for treatment of pancreatic carcinomas, soft tissue sarcomas, testicular tumors, lymphomas, thymomas, Wilms' tumors, renal carcinomas, melanomas, lung tumors, intracerebral metastases, tumors in the head and neck area and/or mammary carcinomas the method comprising combining 1-(2-chloroethyl)-1-nitroso-3-(2-hydroxyethyl)urea (HECNU) and a pharmaceutically acceptable carrier in a unit dosage form.

3. The method according to claim 1, wherein the patient is in need of treatment of pancreatic carcinomas.

4. The method according to claim 1, wherein the patient is in need of treatment of lymphomas.

5. The method according to claim 1, wherein the patient is in need of treatment of melanomas.

6. The method according to claim 1, wherein the patient is in need of treatment of head and neck tumors.
